(19) United States
(12) Patent Application Publication
(10) Pub. No.: US 2010/0273661 A1
(43) Pub. Date: Oct. 28, 2010

(54) Methods and Kits for Predicting Treatment Response in Type II Diabetes Mellitus Patients

(75) Inventors: Yang Qiu, Cary, NC (US); Dilip Rajagopalan, Newtown Square, PA (US); Lei Zhu, Durham, NC (US); Susan C Connor, Middlesex (GB); Guanghui Hu, Phoenixville, PA (US); David Maclean, Middletown, RI (US); Doris Damian, West Newton, MA (US); Amir Handzel, Watertown, MA (US); Rajalakshmi Balasubramanian, Wayland, MA (US)

Correspondence Address: HOWSON & HOWSON LLP, 501 OFFICE CENTER DRIVE, SUITE 210, FORT WASHINGTON, PA, 19034, US

(73) Assignees: SmithKline Beecham Corporation, Philadelphia, PA (US); BG Medicine Inc, Waltham, MA (US)

(21) Appl. No.: 12/663,311

(22) PCT Filed: Jun. 11, 2008
(86) PCT No.: PCT/US08/07304

§ 371 Date: Jul. 1, 2010

Related U.S. Application Data
(60) Provisional application No. 60/934,661, filed on Jun. 15, 2007.

(51) Int. Cl. G01N 33/92; G01N 33/68; G01N 33/48; C12Q 1/37; G01N 33/66; G01N 33/72; C40B 30/00; G06F 19/00; A61B 5/00

(52) U.S. Cl. 506/7; 436/86; 436/129; 436/89; 436/131; 436/71; 435/23; 436/95; 436/94; 436/67; 702/19; 705/3

(57) ABSTRACT

A method for predicting treatment response of a type II diabetes patient to rosiglitazone is provided. The method involves at least one sample from a patient having type II diabetes and analyzing biomarkers predictive of a patient who will respond to treatment with rosiglitazone. The biomarkers include, at least, interleukin-8, histidine, citrate. These biomarkers are identified in at least one classification analyses selected from the group consisting of a majority-vote based classifier and support-vector machine (SVM) classifier. Also provided is a method for predicting treatment response of a type II diabetes patient to glyburide at 8 weeks post-initiation of therapy. The method involves obtaining a sample from a type II diabetes patient who has been treated with glyburide for about 4 weeks and analyzing biomarkers predictive of a patient who will respond to treatment with glyburide at 8 weeks. The biomarkers useful in this method include, at least, sphingomyelin 23:1 and L-phenylalanine. Also provided are kits useful for the methods of the invention.

### BACKGROUND OF THE INVENTION

Various technologies have been applied in recent years to develop predictive biomarkers. For example, gene expression profiling and proteomics have been used to predict the clinical outcome of cancer treatments. See, e.g., Van't Veer, L. J. et al. Nature 415, 530-536 (2002); Ma, X. J. et al. Cancer Cell 5, 607-616 (2004); Raponi, M. et al. Cancer Res. 66, 7466-7472 (2006); Meyerson, M. & Carbone, D., J. Clin. Oncol. 23, 3219-3226 (2005); and Petricoin, E. F. et al. Lancet 359, 572-577 (2002). However, while the analysis and interpretation of the complex data obtained using these techniques is potentially fertile, it is highly challenging.

Among the goals of systems biology is achieving a broad or ‘systematic’ view of biological changes in a cell or organism as a function of some perturbation. This can be assessed by measuring changes in levels of genes, transcripts, proteins or metabolites and mining these changes using intensive multivariate statistics and pattern analyses. The complex nature of the experimental data and computational results also have the potential to more robustly characterize inter-individual relationships between genetic and state variations, and the mechanisms underlying these differences. Similarly, a broad array of measurements might provide greater prognostic ability regarding experimental outcomes as compared to a single biomarker. In drug discovery, these studies can be used to identify candidate biomarkers (or a fingerprint) for a disease, drug efficacy and toxicity. Type II diabetes mellitus (T2DM) represents an interesting case study as it is a multi-factorial disease state with considerable inter-individual heterogeneity.

T2DM is a complex disturbance of physiologic mechanisms affecting many metabolic homeostatic processes, including energy and lipid metabolism, inflammation, clotting and vascular endothelial functions. [Laakso, M., Semin. Vasc. Med. 2, 59-66 (2002); Bastard, J. P. et al., Eur. Cytokine Netw. 17, 4-12 (2006); Ziegler, D., Curr. Mol. Med. 5, 309-322 (2005)]. These disturbances arise from reduced insulin action in peripheral tissues predominantly from a resistance to circulating insulin, together with impaired pancreatic insulin secretion [Kilpatrick, E. S., Diabet. Med. 14, 819-831 (1997)]. Given the causal relationship between hyperglycemia and diabetic complications, measures of glycemia, such as fasting plasma glucose (FPG), glycosylated hemoglobin (HbA1c), or less commonly fructosamine, are typically used to monitor disease progression and treatment efficacy. However, these measures generally do not discriminate between the various pathophysiological phenotypes of diabetes [Petersen, J. L. & McGuire, D. K. Diab. Vasc. Dis. Res. 2, 9-15 (2005); Ostenson, C. G. Acta Physiol Scand. 171, 241-247 (2001)]. For example, patients with T2DM represent a spectrum of states of increased insulin resistance and/or impaired insulin secretory capacity, each with diverse molecular and tissue-specific mechanisms. Understanding the pathophysiologic profile may better inform us of biologic mechanisms and therapeutic efficacy for particular pharmacologic agents.

Commonly used oral antidiabetic agents include the sulfonylurea glyburide, the biguanide metformin, and the thiazolidinedione rosiglitazone, representing a broad range of mechanism of action [Bastard, et al, cited above; Ahmann, A. J. & Riddle, M. C. C, Postgrad. Med. 111, 32-40, 43 (2002)]. Sulfonylureas work primarily by stimulating insulin secretion by binding to sulfonylurea receptors (SUR1 or SUR2) in the pancreatic beta-cell [Gribble, F. M. & Reimann, F. Diabetologia 46, 875-891 (2003)]. Metformin is thought to activate AMP-activated protein kinase (AMPK) and to lower blood glucose primarily by reducing hepatic glucose production [Musi, N. & Goodyear, L. J., Endocrine. 29, 73-80 (2006)]. Rosiglitazone, a member of the thiazolidinedione class of peroxisome proliferator-activated receptor (PPAR)-γ agents, acts primarily in adipose tissue and improves insulin sensitivity in liver and muscle [Vasudevan, A. R. & Balasubramanyam, A., Diabetes Technol. Ther. 6, 850-863 (2004)].

A challenge in diabetes clinical trials and treatment is to more optimally tailor individual drug assignment to the patient's disease stage and underlying pathophysiology.

### SUMMARY OF THE INVENTION

The present invention provides a method for predicting treatment response of a type II diabetes patient to rosiglitazone or to glyburide. This invention allows treatment to be tailored to a patient's pathophysiological phenotype of diabetes and improve the patient's clinical response rate.

In one aspect, the invention comprises obtaining at least one sample from a patient having type II diabetes and analyzing the sample for biomarkers predictive of a patient who will have an increased or decreased likelihood of a response to treatment with a thiazolidinedione, for example, rosiglitazone, wherein the biomarkers are identified in at least one classification analysis selected from the group consisting of a majority-vote classifier and a support-vector machine (SVM) classifier. Suitably, the biomarkers are at least one or more of interleukin-8, histidine (methylhistidine), and citrate.

In another aspect, the invention provides a method for predicting treatment response of a type II diabetes patient to a sulfonylurea, for example, glyburide at some time post-initiation of therapy, for example, at about 8 weeks post-initiation of therapy. The method comprises obtaining a sample from a type II diabetes patient who has been treated with glyburide for about 4 weeks and analyzing the sample for biomarkers predictive of a patient who will have an increased or decreased likelihood of a response to treatment with a sulfonylurea, for example, glyburide at 8 weeks, wherein the biomarkers are identified in at least one of the classification analyses selected from the group consisting of a regression-based classifier, a centroid classifier, a support vector machine (SVM), and a majority-vote-based classifier. Suitably, the biomarkers are at least one or more of phenylalanine and 23:1 sphingomyelin.

In yet another aspect, the invention provides a kit useful for predicting a type II diabetes patient response to rosiglitazone. Such a kit comprises one or more reference standards providing baseline levels of selected biomarker analytes in type II diabetes patients which are responsive to rosiglitazone, and optionally, one or more reference standards providing baseline levels of the selected analytes in type II diabetes patients which are non-responsive to rosiglitazone.

In yet another aspect, the invention provides a kit useful for predicting a type II diabetes patient response to glyburide. Such a kit comprises one or more reference standards providing levels of selected biomarker analytes in type II diabetes patients which have been treated for 4 weeks and are responsive to glyburide, and optionally, one or more reference standards providing levels of the selected analytes in type II diabetes patients which have been treated for 4 weeks and are non-responsive to glyburide.

In a further aspect, the invention provides a method of treatment including predicting a subject's responsiveness to a thiazolidinedione or a sulfonylurea and recommending, authorizing or administering the thiazolidinedione or sulfonylurea if the subject is identified as having an increased likelihood of a desirable response to the thiazolidinedione or sulfonylurea, or declining to recommend, to authorize, or to administer the thiazolidinedione or sulfonylurea unless the subject is identified as having an increased likelihood of a desirable response to the thiazolidinedione or sulfonylurea.

In another aspect, the invention provides a method of predicting a subject's responsiveness to a thiazolidinedione or sulfonylurea including calculating, based on a concentration of at least one biomarker in a sample from a subject, an index having a value indicative of the likelihood of the subject responding to the thiazolidinedione or sulfonylurea and displaying, transmitting or storing the index.

These and other advantages of the invention will be readily apparent from the detailed description of the invention.

### BRIEF DESCRIPTION OF THE FIGURES

FIG. 1 shows workflow of building classifier and model validation. The same workflow was applied to all five methods: Random Forest, Prediction Analysis of Microarray, Partial Least Squares-Discriminant Analysis, Support Vector Machine, T-test/Majority Vote. Samples were divided into a training set and a holdout set. The classifier was built in a 4 fold cross validation (CV) where the optimal number of features used in the classifier was selected to give the best cross validation accuracy. The model was then validated through two procedures. One was holdout prediction, since the holdout set had never been used to build model or classifier. The second procedure was the permutation procedure. The cross validation was repeated for 100-1000 runs (method dependant) with randomized class labels. The percentage of permutation was the percent of permutation runs that had better CV accuracy than the original CV accuracy.

FIGS. 2A-2C show principal component analysis (PCA) plots of selected biomarkers in subjects treated with rosiglitazone, glyburide or metformin. FIG. 2A shows the baseline levels of all 1735 analytes in responders (black circles) and non-responders (white circles). FIG. 2B shows baseline levels of 14 analytes selected using 4 or more classifiers predictive of treatment response across all 3 drugs. FIG. 2C shows baseline levels of 3 conventional markers: glucose, fructosamine, and HbA1c. In each of FIGS. 2A, 2B and 2C, the black circles correspond to responders, and the white circles to non-responders.

FIGS. 3A-3C show PCA plots of selected biomarkers in subjects treated with rosiglitazone. FIG. 3A shows the baseline levels of all 1306 analytes in responders (black circles) and non-responders (white circles). FIG. 3B shows baseline levels of 3 analytes selected using 5 classifiers predictive of treatment response for rosiglitazone-treated subjects. FIG. 3C shows baseline levels of 3 conventional markers: glucose, fructosamine, and HbA1c. In each of FIGS. 3A, 3B and 3C, the black circles correspond to responders and the white circles to non-responders.

FIGS. 4A-4C show the measure of selected biomarkers in urine or serum. FIG. 4A shows urine citrate measured by NMR in rosiglitazone responders (R) and non-responders (N) at week 0 and week 8. FIG. 4B shows serum methyl histidine in rosiglitazone responders and non-responders at week 0 and week 8. FIG. 4C shows serum interleukin-8 (IL-8) in rosiglitazone responders and non-responders at week 0 and week 8. n=9 for responders and n=12 for non-responders.

FIG. 5 shows a scatter plot of serum L-phenylalanine and serum 23:1 sphingomyelin (SM) measured at 4 weeks (after being adjusted for week 0 baseline values and univariate scaled) that are predictive of treatment response at 8 weeks for glyburide-treated subjects. The black circles in the figure correspond to responders, and the white circles to non-responders.

### DETAILED DESCRIPTION OF THE INVENTION

The present invention provides a method for designing and tailoring a course of therapy to a patient with type 2 diabetes mellitus (T2DM). The method of the invention may be used alone, or in addition to, to standard laboratory parameters and clinical decision to increase the speed and likelihood of patient response to the therapy.

Specifically, serum or plasma and urine samples from patients with type 2 diabetes mellitus (T2DM) are measured for specific analytes at baseline (pre-treatment) or at some time after initiating treatment, for example, after 4 weeks of treatment. Such analytes are predictors of a significant treatment response after 8 weeks for a sulfonylurea or a thiazolidinedione antidiabetic agent.

One such thiazolidinedione is 5-[[4-[2-(methyl-pyridin-2-yl-amino)ethoxy]phenyl]methyl]thiazolidine-2,4-dione, also known as rosiglitazone or rosiglitazone maleate [commercially available from GlaxoSmithKline as Avandia®]. See, U.S. Pat. Nos. 5,002,952; 5,741,803; 6,288,095.

Sulfonylureas have been described for use as oral anti-diabetic agents. One such sulfonylurea has the chemical name, 5-chloro-N-[2-[4-(cyclohexylcarbamoylsulfamoyl)phenyl]ethyl]-2-methoxy-benzamide is known under the generic name glyburide or glibenclamide. Glyburide is available commercially under the names Diabeta®, Glynase®, Micronase®. See, also, U.S. Pat. Nos. 3,426,067; 3,454,635; 3,507,961; 3,507,954; 3,979,520; 4,060,634; and 6,830,760, and US Published Application No. US 2001 0036479, for a discussion of glyburide compositions and formulations.

In one embodiment, three analytes, measured at baseline, are associated with response to the thiazolidinedione rosiglitazone after eight weeks of treatment and are biomarkers thereof. Two analytes, measured at 4 weeks, were found to be early therapy indicators of effective 8 week response to the sulfonylurea glyburide. In one embodiment, these analytes are detected in serum or urine using multivariate classification techniques.

A variety of multivariate classification are known in the art. Particularly desirable techniques described herein include RandomForest (RF)™, Prediction Analysis for Microarrays (PAM), Partial Least Squares-Discriminant Analysis (PLS-DA), Support Vector Machines (SVM), and T-test classifier.

As defined herein, RandomForest (RF), RF is a decision-tree-based classifier that is constructed using an algorithm originally developed by Leo Breiman [Breiman L, “Random forests,” Machine Learning 2001, 45:5-32]. The classifier uses a large number of individual decision trees and decides the class by choosing the mode of the classes as determined by the individual trees. The individual trees are constructed using the following algorithm: (1) Assume that the number of cases in the training set is N, and that the number of variables in the classifier is M; (2) Select the number of input variables that will be used to determine the decision at a node of the tree; this number, m should be much less than M; (3) Choose a training set by choosing N samples from the training set with replacement; (4) For each node of the tree randomly select m of the M variables on which to base the decision at that node; (5) Calculate the best split based on these m variables in the training set.

As used herein, Prediction Analysis for Microarrays (PAM) is a centroid classifier proposed by Narashiman, “Diagnosis of multiple cancer types by shrunken centroids of gene expression,” PNAS 2002 99:6567-6572. PAM computes a standardized centroid for each class which is the average analyte value in each class divided by the within-class standard deviation for the analyte. Nearest centroid classification takes the analyte profile of a new sample, and compares it to each of these class centroids. The class whose centroid that it is closest to, in squared distance, is the predicted class for that new sample. Nearest shrunken centroid classification makes one important modification to standard nearest centroid classification. It “shrinks” each of the class centroids toward the overall centroid for all classes by an amount known as the threshold. This shrinkage consists of moving the centroid towards zero by threshold, setting it equal to zero if it hits zero. For example, if threshold was 2.0, a centroid of 3.2 would be shrunk to 1.2, a centroid of −3.4 would be shrunk to −1.4, and a centroid of 1.2 would be shrunk to zero. After shrinking the centroids, the new sample is classified by the usual nearest centroid rule, but using the shrunken class centroids. This shrinkage has two advantages: 1) it can make the classifier more accurate by reducing the effect of noisy analytes, 2) it does automatic feature selection. In particular, if a feature is shrunk to zero for all classes, then it is eliminated from the prediction rule. Alternatively, it may be set to zero for all classes except one, and this class is then distinguished by high or low value for that analyte. This threshold value is the free parameter for classifier and is determined via cross-validation as described below.

As used herein Partial Least Squares-Discriminant Analysis (PLS-DA) is a regression-based classification method that originated in social sciences [Wold, H. (1966). Estimation of principal components and related models by iterative least squares. In P. R. Krishnaiaah (Ed.). Multivariate Analysis. (pp. 391-420) New York: Academic Press] and became popular in Chemometrics due to Svante Wold [Geladi & Kowalski, (1986) Partial least square regression: A tutorial. Analytica Chemica Acta, 35, 1-17]. PLS regression is analogous to Principal Components Analysis (PCA) which is a projection technique to reduce multidimensional data to the few most important dimensions that can explain the most variation in the data. PLS regression finds components of the independent variable space that are relevant to the outcome space. PLS regression searches for a set of components (called latent vectors) that performs a simultaneous decomposition of dependent and independent variable spaces with the constraint that these components maximize the covariance of the two spaces.

As used herein, Support Vector Machines (SVM), is a method to separate different classes of samples in multidimensional space using hypersurfaces. In the simplest case, these surfaces are hyperplanes (linear separators). More complex separators can be applied using kernel functions. Among the possible separators, SVM selects the one where the distance of the separator from the closest data points is as large as possible. A kernel function is used to map the original data into feature space where they become separable. Radial basis functions (RBF) were used in this analysis. RBF is one of the widely used kernel functions. X and g are parameters of this basis function, along with the number of analytes in the classifier. These 3 parameters were determined via cross-validation. Prior to building the SVM, appropriate features must be selected, and the t-test was used in this work.

As used herein, T-test classifier is a simple, majority-vote-based classifier that uses a t-test for feature selection. This method is somewhat similar to PAM, but the prediction rule is more interpretable. This method is only applicable to 2-group classification problems. The first step in this classifier is to perform a t-test between the two sample groups and generate a list of analytes ordered in decreasing order of t-test significance. For each analyte, the mean value in both sample groups is calculated. The next step is to calculate a threshold value for each analyte which is the mean value of the two means calculated above. For equally sized sample groups, this threshold value is simply the overall mean value of the analyte. Each analyte can then be used independently to classify a sample, depending on which side of the threshold the analyte value for that sample lies. The only free parameter of this classifier is the number of analytes in the classification rule, and this is determined via cross-validation as described below. For a t-test classifier with N analytes, a prediction for each sample is made independently using all N analytes, and the overall prediction is made by majority vote. In case of ties when N is even, the prediction using the most significant analyte is used.

These multivariate classification methods may be used alone, or in combination with other analysis methods, in the method of the invention.

In one embodiment, the invention provides a method for predicting treatment response of a type II diabetes patient to a thiazolidinedione, for example, rosiglitazone. The method involves obtaining at least one sample from a patient having type II diabetes and analyzing the biomarkers predictive of a patient who will have an increased or decreased likelihood of a response to treatment with the thiazolidinedione, for example, rosiglitazone. The biomarkers predictive of an increased or decreased likelihood of a response to thiazolidinedione include citrate, methylhistidine and interleukin-8. These biomarkers are identified in at least one classification analyses selected from the group consisting of a majority-vote classifier and a support-vector machine (SVM) classifier. Suitably, the biomarkers are identified in both a majority-vote classification analysis and a support-vector machine classification (SVM) analysis.

In one embodiment, the biomarkers include urine citrate, serum or plasma interleukin-8 and serum or plasma histidine (e.g., methylhistidine). Optionally, the sample(s) may be analyzed for additional biomarkers, e.g., such as those selected from the group consisting of lactate, glycerol, leptin, interleukin-12 (IL-12) p40, plasminogen activator inhibitor (PAI)-1, total free fatty acid, insulin, insulin growth factor (IGF)-1, PPAP-A, total TG, glycerol, and amino acids.

In one desirable embodiment, the invention provides a method for predicting treatment response of a type II diabetes patient to rosiglitazone by analyzing biomarkers from a pre-treated patient (i.e., a patient not previously treated with rosiglitazone) having type II diabetes comprising at least one or more of serum interleukin-8, serum histidine and urine citrate, said biomarkers identified in at least a majority-vote classification analysis and a support vector machine (SVM) classification analysis. These biomarkers have been found to be at least about 80% predictive of response at 8 weeks for a patient prior to rosiglitazone treatment. The biomarkers may be further analyzed in one or more additional classification analysis selected from the group consisting of a centroid classifier, a regression-based classifier, and a tree-based classifier.

In one embodiment, serum IL-8 concentrations are higher in patients who have an increased likelihood of a desirable response to the thiazolidinedione, for example, to rosiglitazone as compared to non-responders.

In another embodiment, serum histidine concentrations are higher in patients who have an increased likelihood of a desirable response to the thiazolidinedione, for example, to rosiglitazone as compared to non-responders.

In a further embodiment, urine citrate concentrations are lower in patients who have an increased likelihood of a desirable response to the thiazolidinedione, for example, to rosiglitazone as compared to non-responders.

In another embodiment, the invention provides a method of treatment including predicting a subject's responsiveness to a thiazolidinedione as described above and recommending, authorizing or administering the thiazolidinedione if the subject is identified as having an increased likelihood of a desirable response to the thiazolidinedione.

In a related embodiment, the invention provides a method of treatment including predicting a subject's responsiveness to a thiazolidinedione as described above and declining to recommend, to authorize, or to administer the thiazolidinedione unless the subject is identified as having an increased likelihood of a desirable response to the thiazolidinedione.

In yet another embodiment, the invention provides a method for predicting treatment response of a type II diabetes patient to a sulfonylurea, for example, glyburide, post-initiation of therapy, for example, at 8 weeks post-initiation of therapy. The method involves obtaining a sample from a type II diabetes patient who has been treated with glyburide, for example, for about 4 weeks and analyzing the sample for biomarkers predictive of a patient who has an increased or decreased likelihood of a response to treatment with the sulfonylurea, for example, glyburide at 8 weeks. The biomarkers predictive of a response to sulfonylurea include phenylalanine and 23:1 sphingomyelin. In one embodiment, the biomarkers are identified in at least one of the classification analyses selected from the group consisting of a regression-based classifier, a centroid classifier, a support vector machine (SVM), and a majority-vote-based classifier. In another embodiment, the biomarker is identified in the majority-vote-based classifier. In another embodiment, at least two of the classification analyses are used. In another embodiment, at least three of the classification analyses are used.

In one embodiment, the biomarkers are, at least, one or more of serum or plasma sphingomyelin 23:1 and L-phenylalanine. Optionally, additional analytes may be included in the analysis, including, e.g., glucose, fructosamine and HbA1c.

In one embodiment, the regression-based classifier is a partial least squares-discriminant analysis (PLS-DA). In another embodiment, the centroid classifier is a prediction analysis for microarrays. The majority-vote-based classifier can be a t-test.

In another embodiment, the invention provides a method of treatment including predicting a subject's responsiveness to a sulfonylurea as described above and recommending, authorizing or administering the sulfonylurea if the subject is identified as having an increased likelihood of a desirable response to the sulfonylurea.

In a related embodiment, the invention provides a method of treatment including predicting a subject's responsiveness to a sulfonylurea as described above and declining to recommend, to authorize, or to administer the sulfonylurea unless the subject is identified as having an increased likelihood of a desirable response to the sulfonylurea.

In still a further embodiment, the invention provides a kit useful for predicting a type II diabetes patient response to a drug selected from the group consisting of a thiazolidinedione, for example, rosiglitazone or a sulfonylurea, for example, glyburide. Such a kit may contain, e.g., one or more reference standards providing baseline levels of selected biomarker analytes in type II diabetes patients which are responsive to rosiglitazone, and optionally, one or more reference standards providing baseline levels of the selected analytes in type II diabetes patients which are non-responsive to a drug selected from rosiglitazone. In another embodiment, such a kit may contain, e.g., one or more reference standards providing levels of selected biomarker analytes in type II diabetes patients which have been treated with a sulfonylurea for 4 weeks and which are responsive to the sulfonylurea, and optionally, one or more reference standards providing levels of the selected analytes in type II diabetes patients treated with a sulfonylurea for 4 weeks and which are non-responsive to the sulfonylurea (e.g., glyburide).

According to various embodiments of the invention, the levels or concentrations of one or more of the biomarkers are measured as absolute concentrations, relative concentrations, or as a comparison of the absolute concentration or the relative concentration of one or more of the biomarkers to a value indicative of the likelihood of the response. According to one embodiment, the value is a threshold distinguishing populations having differing likelihoods of the response.

In another embodiment, the invention provides a method of predicting a subject's responsiveness to a thiazolidinedione, for example, rosiglitazone, including calculating, based on a concentration of at least one biomarker in a sample from a subject, an index having a value indicative of the likelihood of the subject responding to the thiazolidinedione and displaying, transmitting or storing the index. According to this embodiment, the biomarkers include one, two or three of citrate, methyl histidine and interleukin-8.

In a related embodiment, the invention provides a method of predicting a subject's responsiveness to a sulfonylurea, for example, glyburide, including calculating, based on a concentration of at least one biomarker in a sample from a subject, an index having a value indicative of the likelihood of the subject responding to the sulfonylurea and displaying, transmitting or storing the index. According to this embodiment, the biomarkers include one or both of phenylalanine and 23:1 sphingomyelin.

According to these embodiments, the concentration can be a relative concentration. The index can be calculated based on the concentrations of methyl histidine and interleukin-8 in a blood-based sample and the concentration of citrate in a urine sample. Further, the index can be displayed on a screen or a tangible medium. The index can be transmitted to a person in a medical industry, to a medical insurance provider or to a physician. The index can be transmitted prior to the medical insurance provider or the physician approving the thiazolidinedione or the sulfonylurea for the subject.

According to additional embodiments, the subject is a human or a non-human mammal. Further, the subject can be diabetic or non-diabetic.

### EXAMPLES

The study described herein was relatively small-scale (75 male subjects with T2DM) and short-term (8 weeks of treatment). Serum and urine samples were obtained at pre-treatment baseline, and after 4 and 8 weeks of treatment with one of the following: placebo, rosiglitazone, metformin or glyburide. High information content nuclear magnetic resonance (NMR) and liquid chromatography/mass spectroscopy (LC/MS)-based metabolomic platforms, including polar metabolite and lipid profiling, were used to profile the serum and urine samples. A variety of multivariate analysis techniques were used to determine whether polar low molecular weight metabolites, lipids, or fatty acids, analyzed in readily accessible fluids can be used to predict drug responder status at week 8 based on their measurement at baseline or at week 4.

### Example 1

### Experimental Design

Male subjects aged 30 to 70 years with a documented history of stable T2DM for no more than 10 years duration were eligible for the study described herein if they had been previously treated with diet and exercise alone, monotherapy or low-dose combination therapy. Fasting plasma glucose (FPG) at screening could not exceed 225 mg/dL for subjects treated with diet and exercise alone or 180 mg/dL for subjects receiving monotherapy or low-dose combination therapy. HbA1c was required to be within 5.7 to 10.0% with the following conditions; subjects with HbA1c between 5.7 and 9% must have been diabetic for less than 5 years and treated with mono or low dose combination therapy and have a FBG of 125 to 180 mg/dL, and subjects with HbA1c between 9.1 and 10.0% must not have been treated with combination therapy. In addition, body mass index must have been within the range of 25 to 37.5 kg/m², for subjects aged 35-55 years, or 25 to 35.0 kg/m² for subjects aged 56 to 70 years. Use of insulin for greater than 7 days during the 6 months prior to screening was prohibited and use of the following medications within 1 month prior to screening that may affect response of experimental drugs was also prohibited: thiazolidinediones, high dose HMG-CoA reductase inhibitors (statins), and high dose cholesterol absorption inhibitors. Eligible subjects entered the treatment phase after a five week washout period and were randomly assigned to one of four single-blind treatment groups: 19 to placebo, 22 to rosiglitazone, 21 to metformin, and 21 to glyburide. All subjects were blinded to study medication (single-blinded). Based on glucose levels, doses of glyburide (total dose 5 to 15 mg) and metformin (total dose 500 to 1500 mg) were single-blind titrated upwards at weeks 2 and 4, and rosiglitazone was titrated from 2 mg twice daily to 4 mg twice daily at week 4 only. Blood and urine samples were collected prior to and at 4 and 8 weeks following initiation of treatment. The baseline (week 0) clinical and biochemical characteristics of participants are shown in the Table 1.

TABLE 1
Baseline (week 0) clinical and biochemical
characteristics of patients.

| | | | | NORMAL | NORMAL |
|---|---|---|---|---|---|
| | Units | Mean | SD | HIGH | LOW |
| Fructosamine | μmol/L | 305.1 | 52.8 | | |
| Triglycerides | mg/dL | 185.5 | 104.5 | 213 | 44 |
| Free fatty acid | mEq/L | 0.5 | 0.2 | 0.6 | 0.1 |
| Glucose | mg/dL | 165.5 | 43.5 | 115 | 70 |
| Insulin | μlU/mL | 11.0 | 7.2 | 23 | 1.9 |
| Glycosylated | % | 7.2 | 0.9 | | |
| haemoglobin | | | | | |
| Body Mass Index | kg/m2 | 30.3 | 3.2 | | |
| Age of the Patient | year | 56.0 | 8.2 | | |
| Diastolic blood | mm Hg | 79.0 | 8.7 | | |
| pressure | | | | | |
| Systolic blood pressure | mm Hg | 126.5 | 12.1 | | |
| Waist Circumference | cm | 104.9 | 9.1 | | |
| Weight | kg | 94.4 | 12.7 | | |
| Duration of diabetes | year | 3.9 | 2.8 | | |

A. Data Generation

Serum and urine samples were analyzed using various metabolomic platforms and with traditional serum biomarker (“non-omic”) measurements. Both urine and serum were measured by nuclear magnetic resonance (NMR)-based metabolic profiling. Serum samples were also analyzed by liquid chromatograph (LC)/mass spectrometry (MS) for polar metabolites and lipids, and gas chromatograph (GC)-flame ionization for fatty acids (lipidomics). Analysis of clinical chemistry, serum and plasma protein biomarkers, and physiological parameters such as body weights were also included in the data set. In total, there were over 3000 variables included in the analysis: 98 analytes from clinical chemistry, 303 fatty acids from GC-flame ionization, 467 lipids from LC/MS, 921 LC/MS polar metabolites, 314 NMR serum metabolites, and 1006 urine NMR metabolites which include both 0 hr and 6 hr measurements (urine samples were collected at both 0 hour and 6 hours). Both the details of the metabolomics platform data acquisition and signal processing can be referred to published review [Listgarten, J. & Emili, A. Statistical and computational methods for comparative proteomic profiling using liquid chromatography-tandem mass spectrometry. Mol. Cell Proteomics. 4, 419-434 (2005)].

B. Data Preprocessing

For the data collected at week 4, analysis of covariance (ANCOVA) outlier-removed and adjusted data was used [Steel, R. Principles and Procedures of Statistics: A Biometrical Approach. eds. Torrie, J. & Dickey, D. 3rd edition. 1996. McGraw-Hill Companies]. Due to the effect of covariate and multiple design factors, studentized residuals from the ANCOVA were used to define outliers. The cutoff for outliers was chosen based on the knowledge of biological variation or experimental outliers, which was 3 standard deviations for non-omic analytes and urine NMR, and 2 for all the other platforms. Less than 5% of data was removed as outliers in each treatment group. In order to reduce variability in the data caused by nuisance factors, ANCOVA residuals were used to adjust week 4 data to correct for individual subject variation at week 0, prior therapy and concomitant medications. Further data preprocessing addressed missing values, since several multivariate classification methods do not allow missing values. Metabolic analytes with too many missing values were eliminated. Up to 25% missing values in either class were allowed for non-omic analytes, up to 20% missing allowed for serum NMR data, and up to 15% missing data was allowed for the remaining platforms. For training subjects, missing values were set to the median value of non-missing training subjects in same class.

For holdout subjects, missing values were set to the median value of all non-missing subjects. After data preprocessing, there were about 1500 analytes remaining for use in classification. The final step was location and scale transformation performed across all samples in the analysis to ensure the samples were from the same distribution and comparable to each other.

C. Multivariate Classification Methods

Analysis of large volumes of data with a high number of variables (dimensions) poses a challenge for data classification. The five classification methods used were Random Forest (RF), Prediction Analysis for Microarray (PAM), Partial Least Square-Discriminant Analysis (PLS-DA), Support Vector Machines (SVM), and T-test/Majority Vote (Ttest). RF is a decision tree-based classifier using an algorithm originally developed by Leo Breiman [Breiman, L. Random Forests. Machine. Learning 45, 5-32 (2001)]. It grows many classification trees (forest) and the forest chooses the classification of a sample by choosing the class that has the most votes across all trees. Software for performing this method is available from Salford Systems. PAM is a centroid classifier proposed by Narashiman which computes a standardized centroid for each class and predicts the class of a new sample based on the its distance to the class centroid [Tibshirani, R., Hastie, T., Narasimhan, B., & Chu, G. Diagnosis of multiple cancer types by shrunken centroids of gene expression. Proc. Natl. Acad. Sci. U.S.A 99, 6567-6572 (2002)]. [software for PAM is available from Stanford University]. PLS regression is analogous to Principal Components Analysis (PCA), which is a projection technique to reduce multidimensional data to a set of dimensions that explain the most variation in the data [Hellberg, S., Sjostrom, M., & Wold, S., Acta Chem. Scand. B, 40, 135-140 (1986)]. [Software for PLS available from Camo Software]. SVM is a method to separate different classes of samples in multidimensional space using a hyper-surface that maximizes the geometric margin [Cortes, C. Support-vector networks. ed. Vapnik, V. Machine Learning 20[3], 273-297 (1995)]. [software for SVM available from Cornell University]. Ttest classifier is a simple, majority vote-based classifier that uses a t-test for feature selection. The next step for Ttest classifier is to calculate a threshold value for each selected feature, which is the mean value of the two means from the two classes. Each analyte can then be used independently to classify a sample, depending on which side of the threshold the analyte value for that sample lies and the final class is determined by majority vote. Each method is described in this specification.

D. Building Classifier and Model Validation

Data overfitting is a known issue in data mining where the number of variables greatly exceeds the number of observations. In order to ensure that the classifier has not overfitted the data, proper data validation procedures should be adopted [Radmacher, M. D., McShane, L. M., & Simon, R. A paradigm for class prediction using gene expression profiles. J. Comput. Biol. 9, 505-511 (2002)]. A standard procedure was used for all five classification methods. The samples to be classified were randomly divided into a training set and a holdout set. The training samples were used to determine parameters for each classifier such as the optimal number of analytes to maximize accuracy (based on the percentage of samples correctly classified) using a cross-validation procedure. A four-fold cross-validation (CV) was used in this analysis, where the training samples were randomly divided into 4 CV groups that were as class balanced as possible. In the CV procedure, numerous combinations of free parameters of each classifier were selected to span the parameter space; the classifiers were built using 3 out of the 4 CV groups and the resulting models were used to make class predictions on the samples in the 4th group. The particular combination of parameters that maximized accuracy over the entire parameter space was selected as the optimal parameter set. The accuracy corresponding to this optimal parameter set is known as the CV accuracy. Once the optimal parameter set was determined, the entire set of 4 groups (all the training samples) were used to rebuild the classifier and make class predictions on the holdout set to obtain the holdout accuracy. For individual drug fingerprints, the total number of samples available was only around 20; in these cases, division into training and holdout sets was not performed. All the samples were used in CV mode.

To assess the significance of the CV results, a permutation strategy was adopted. The four-fold CV step was repeated using randomly permuted class labels between 100 and 1000 times depending on the method. Due to the small sample size (n=20-60) and small class number (2 classes), the classifier was considered significant if the percentage of permutation runs with better CV accuracy than the un-permuted case was on the order of 10% or less (p<0.1).

### Example 2

### Definition of Treatment Response (“Responder”) Using a Composite Score of Glycemic-Lowering Efficacy

Originally, efficacy response was defined as a FPG decrease of greater than 30 mg/dl. However, glucose is highly variable and influenced by short-term changes in diet, activity or stress, whereas integrated measures of glycemic response can estimate whether a patient's average glucose has changed over time (weeks to months) in response to treatment [Tahara, Y. & Shima, K. Kinetics of HbA1c, glycated albumin, and fructosamine and analysis of their weight functions against preceding plasma glucose level. Diabetes Care 18, 440-447 (1995)]. Fructosamine, whose half-life is determined by that of albumin, provides a measure of integrated glucose over a period of 2-4 weeks. HbA1c, a form of glycosylated hemoglobin, is the gold standard measure of integrated glucose over a 6-12 week period.

This analysis used an eight week study, which is less than the twelve weeks generally required to reach full glycemic efficacy with PPAR-γ agonists. Consequently it was necessary (for the sake of model building) to derive a surrogate measure of efficacy, one that would reflect a developing response trend. This composite measure of efficacy (described below) was derived solely for its use in the modeling in this study, and has not been tested or validated in a general context.

Three measures of glycemic efficacy—FPG, fructosamine and HbA1c—were used to determine if they could more reliably predict responder status when used in combination. Combined data from 3 larger clinical trials (GSK trial 49653_{—}011, 49653_{—}020, 49653_{—}024, http://ctr.gsk.co.uk/welcome.asp) were used to model changes in FPG, fructosamine and HbA1c at 8 weeks versus measured changes in HbA1c (the accepted gold standard) at 17 weeks. The goal was to establish an efficacy measure and responder criterion at 8 weeks that matches the 17-week “truth”. Many composite scoring rules were able, with 8 week data, to outperform observed change in any single measure in predicting the HbA1c change at 17 weeks. A rule was chosen from within the mathematical ‘space’ of choices that was relatively simple and reflected the perceived relative value of the glycemic markers as discussed above: 1(%ΔFPG)+2(%ΔFructosamine)+1.5(%ΔHbA1c)=response. Thus, if the composite % reduction is greater than 30% using this formula, the subject is classified as a ‘responder’. Using the composite score definition, the fraction of subjects responding in this eight week trial was shown in Table 2 and ranged between 43 to 60% for the 3 treatments.

TABLE 2
Fractions of responders in each treatment after
eight weeks of treatment.

| Placebo | Rosiglitazone | Metformin | Glyburide |
|---|---|---|---|
| 0.13 | 0.43 | 0.53 | 0.60 |

A. Cross-Drug Fingerprint or Individual Drug Fingerprint Prior to Treatment that is Predictive of Eight Week Treatment Response.

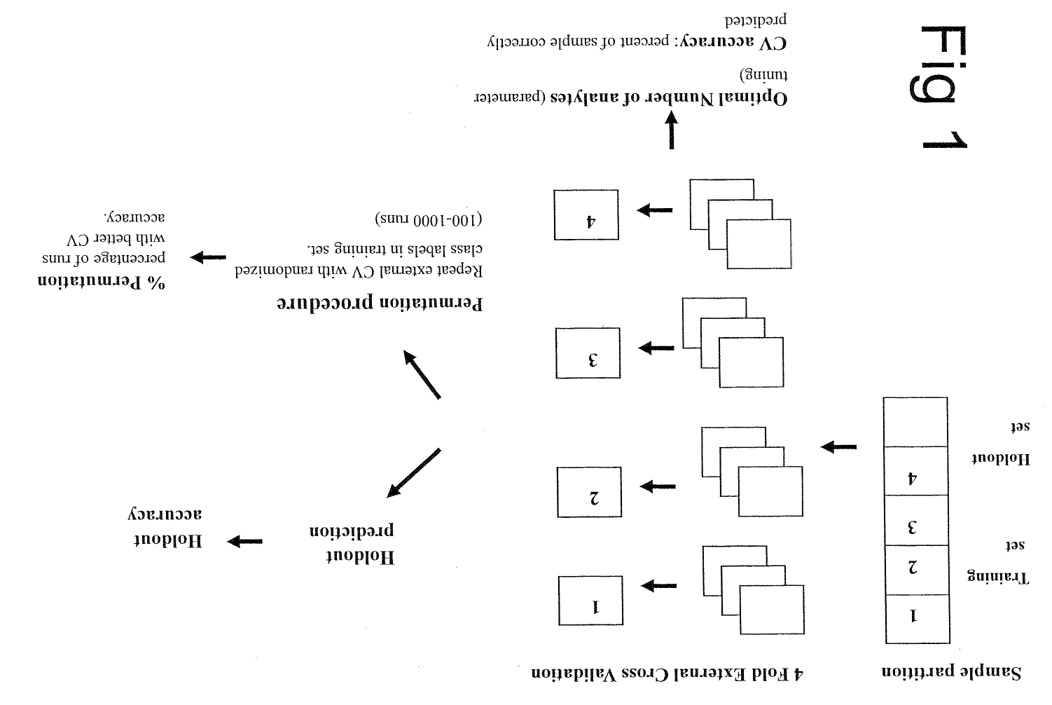

The approach used was to apply five representative classification techniques in parallel for every question of interest and compare results from different methods. Those five methods included both linear and non-linear classification in original space or transformed space. The workflow was kept as consistent as possible (FIG. 1).

For classification using metabolomic data, serum measurements of conventional glycemic markers (FPG, fructosamine and HbA1c) were excluded from the combined dataset, as were all NMR peaks from serum and urine corresponding to glucose. The rationale for exclusion was to identify analytes other than the conventional glycemic markers. Results for baseline prediction of treatment response are discussed below.

(a) Cross-Drug Fingerprint

The goal of this study was to identify a set of analytes that can predict 8 week patient response to oral antidiabetic agents with diverse mechanisms of action. If a classifier could successfully predict treatment response from 3 diverse mechanisms, it could be potentially useful to predict response of a new drug with a different mechanism of action. Classification analysis was applied to data from 60 subjects who were treated with one of the 3 study drugs. The samples were divided into 46 subjects in the training group and 14 in the holdout group. Both treatment type and class were properly balanced in the training and holdout groups. Results from each of the classification methods are summarized in Table 3.

More particularly, in Table 3, the number of analytes indicates the optimal number that maximized prediction accuracy in cross-validation. The percentage of permutation is the percent of permutation runs that had better CV accuracy than the original CV accuracy. The number in brackets indicates the number of permutation runs which was method dependant.

TABLE 3
Cross-drug classification results.

| | Number | CV Accuracy | | | | % Permutation | | Holdout Accuracy | | | |
|---|---|---|---|---|---|---|---|---|---|---|---|
| | of | (n = 46) | | | | (# of | | (n = 14) | | | |
| Method | analyte | R | M | G | T | permutation) | | R | M | G | T |
| PLS-DA | 190 | 56 | 67 | 53 | 59 | 5.6 | (500) | 60 | 75 | 60 | 64 |
| RF | 20 | 94 | 87 | 73 | 85 | <1 | (100) | 60 | 100 | 40 | 64 |
| PAM | 138 | 69 | 80 | 53 | 67 | 9 | (100) | 60 | 50 | 40 | 50 |
| SVM | 5 | 81 | 73 | 53 | 70 | 7 | (100) | 40 | 75 | 20 | 43 |
| Ttest | 75 | 62 | 87 | 73 | 74 | 3.7 | (1000) | 80 | 100 | 41 | 71 |

R = rosiglitazone. M = metformin, G = glyburide, T = overall accuracy.

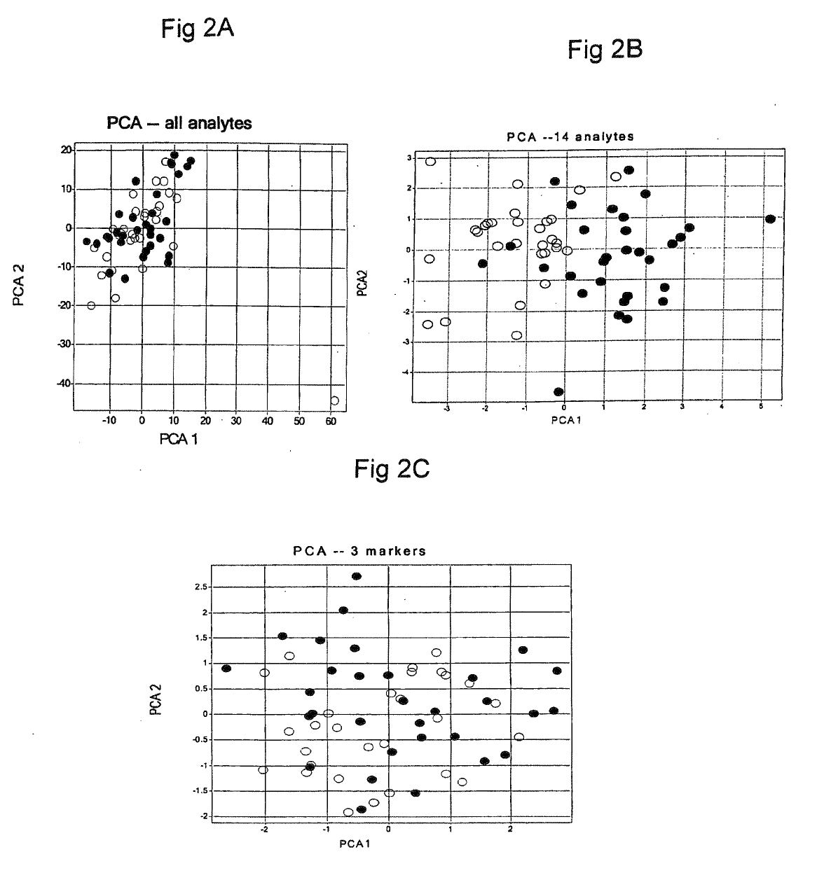

The cross-validation (CV) accuracy across five classification methods ranged from 59 to 74%. The permutation procedure indicated that when cross validation was repeated with a randomized class label, no more than 9% (for the Prediction Analysis for Microarray PAM classifier [Tibshirani, R., Hastie, T., Narasimhan, B., & Chu, G. Diagnosis of multiple cancer types by shrunken centroids of gene expression, Proc. Natl. Acad. Sci. U.S.A 99, 6567-6572 (2002)] of the CV accuracy was better than the original CV accuracy; in other words, the permutation p value ranged from 0.09 to less than 0.01 depending on the method. The number of analytes used by each classifier ranged from 5 to 190. Models were validated by predicting the responder status of 14 subjects in a holdout group and the accuracy ranged from 43% to 71%. In particular, the T-test/Majority Vote (Ttest) classifier using 75 analytes gave the best holdout prediction of 71% accuracy. The prediction accuracies were less for glyburide than for metformin and rosiglitazone. In addition, many of the composition patterns of markers chosen for these lists are similar to those for metformin and rosiglitazone evaluated as a binary group (see below). It is evident from the principal component analysis (PCA) plot (FIG. 2B) that the 14 analytes (Table 4) picked by at least 4 classifiers have the ability to discriminate non-responders from responders, while using 11,735 analytes (FIG. 2A) did not separate the two groups.

TABLE 4
Predictive fingerprint at baseline. The number in the method column
indicates how many methods selected the analyte.

| | | |
|---|---|---|
| Platform: Analyte_ID | Annotation | Method |

Cross-drug baseline fingerprint, 14 analytes picked by at least four methods

| | | |
|---|---|---|
| NMR:3.1408 | 1-Methyl-L-Histidine DD (b-CH2′) | 5 |
| Polar:LC-MS-Polar-Metabolites:452_2495 | N/A | 5 |
| LipPerc:Diglyceride_22:6n3 | Diglyceride_22:6n3 | 5 |
| LipQuant:Diglyceride_22:6n3 | Diglyceride_22:6n3 | 5 |
| NMR:2.2704 | L-Valine M (b-CH) | 4 |
| NMR:2.2832 | L-Valine and/or 3-Hydroxybutyric acid | 4 |
| | Other (a-CH2/b-CH2) | |
| Polar:LC-MS-Polar-Metabolites:177_0932 | L-Threonine M + H (M + 1) | 4 |
| Polar:LC-MS-Polar-Metabolites:218_1140 | L-Cysteine Related Adduct (M) | 4 |
| Polar:LC-MS-Polar-Metabolites:228_1822 | d5-Phenylalanine M + 1(M + H) | 4 |
| NMRh0:[6.74 . . . 6.77] | Baseline | 4 |
| NMRh0:[9.26 . . . 9.27] | N/A | 4 |
| NMRh6:[4.37 . . . 4.40] | Baseline | 4 |
| NonOmics:Chloride | Chloride | 4 |
| NonOmics:Sodium | Sodium | 4 |

Metformin baseline fingerprint, 5 analytes picked by both SVM and PLSDA

| | | |
|---|---|---|
| NMR:1.7008 | L-Leucine M (CH2) | 2 |
| Polar:LC-MS-Polar-Metabolites:466_2491 | Sphingosyl-phosphocholine M + 1 (M + H) | 2 |
| Polar:LC-MS-Polar-Metabolites:951_2597 | N/A | 2 |
| NMRh6:[4.45 . . . 4.46] | 0 | 2 |
| LipQuant:Phosphatidylethanolamine_18:3n3 | Phosphatidylethanolamine_18:3n3 | 2 |

Rosiglitazone baseline fingerprint, 24 analytes picked by at least 3 methods

| | | |
|---|---|---|
| HumBio:IL-8 | IL-8 | 5 |
| NMRh6:[2.69 . . . 2.72] | Citrate | 5 |
| NMR:3.1408 | 1-Methyl-L-Histidine DD * | 5 |
| LipPerc:Phosphatidylcholine_22:5n3 | Phosphatidylcholine_22:5n3 | 4 |
| NMRh6:[9.80 . . . 9.82] | Baseline | 4 |
| NMRh6:[1.09 . . . 1.12] | 2-keto-3-methyl-N-valerate * | 4 |
| NMRh0:[6.54 . . . 6.56] | Unassigned | 4 |
| NMRh0:[6.20 . . . 6.22] | unassigned low level peaks | 4 |
| Polar:LC-MS-Polar-Metabolites:411_0854 | NA | 4 |
| NMR:2.3088 | L-Valine and/or 3-Hydroxybutyric acid | 4 |
| | Other (a-CH2/b-CH2) | |
| LipPerc:Phosphatidylcholine_20:2n6 | Phosphatidylcholine_20:2n6 | 3 |
| HumBio:Leptin | Leptin | 3 |
| NMRh6:[9.17 . . . 9.20] | Baseline | 3 |
| NMRh6:[3.59 . . . 3.60] | Unassigned | 3 |
| NMRh6:[1.76 . . . 1.77] | Lysine | 3 |
| NMRh6:[1.26 . . . 1.28] | 3-hydroxyisovalerate | 3 |
| NMRh6:[1.12 . . . 1.13] | low level unassigned peaks | 3 |
| NMRh0:[9.26 . . . 9.27] | Side of N1-methylnicotinamide* | 3 |
| NMRh0:[7.69 . . . 7.72] | Nalpha-Methylhistidine, indoxyl | 3 |
| | sulphate | |
| NMRh0:[6.78 . . . 6.80] | Baseline | 3 |
| NMR:3.6402 | Glycerol Other (CH2) | 3 |
| NMR:2.2704 | L-Valine M (b-CH) | 3 |
| NMR:0.9902 | L-Valine D (CH3) | 3 |

AVANDA ® met baseline fingerprint, 13 analytes picked by at least 3 methods

| | | |
|---|---|---|
| NMR:3.1408 | 1-Methyl-L-Histidine DD (b-CH2′) | 3 |
| Polar:LC-MS-Polar-Metabolites:264_2240 | d3-glutamate M + H(M + 1) | 3 |
| NMR:2.2704 | L-Valine M (b-CH) | 3 |
| Polar:LC-MS-Polar-Metabolites:466_2491 | Sphingosyl-phosphocholine M + H(M + 1) | 3 |
| Polar:LC-MS-Polar-Metabolites:228_1822 | d5-Phenylalanine M + H(M + 1) | 3 |
| Polar:LC-MS-Polar-Metabolites:327_2636 | NA | 3 |
| NMR:2.2896 | L-Valine and/or 3-Hydroxybutyric acid | 3 |
| | Other (a-CH2/b-CH2) | |
| HumBio:Glycerol | Glycerol | 3 |
| LipPerc:Phosphatidylcholine_18:1n7 | Phosphatidylcholine_18:1n7 | 3 |
| LipPerc:Triglyceride_% SFA | Triglyceride_% SFA | 3 |
| NMR:0.92 | L-Isoleucine T (d-CH3) | 2 |
| NMR:2.2832 | L-Valine and/or 3-Hydroxybutyric acid | 2 |
| | Other (a-CH2/b-CH2) | |
| NMRh0:[1.29 . . . 1.32] | lipid and fatty acid acyl side chain | 2 |

Since T2DM is a disease with established biomarkers of disease severity and therapeutic efficacy, it is important to establish whether classification using metabolomic platforms offers any advantage relative to the conventional glycemic biomarkers. Results for prediction of treatment response using only the 3 conventional markers at baseline (FPG, fructosamine, HbA1c) indicated that none of the classifiers yield a statistically significant model (data not shown), suggesting that additional data which more comprehensively represent the underlying biology, such as those acquired using metabolomics, are needed to predict treatment response. A PCA plot using those 3 markers also showed inter-mixed responders and non-responders (FIG. 2C).

Baseline predictions were also made for the 40 subjects treated with either rosiglitazone or metformin. Thirty-one (31) subjects were chosen for the training group, and 9 were set aside as a holdout group. Both treatment type and class were properly balanced in the training and holdout groups. Results of this exercise from each of the classification methods are summarized in Table 5.

TABLE 5
Classification results for Rosiglitazone
or Metformin-treated subjects.

| | Number | CV Accuracy | | | % Permutation | Holdout Accuracy | | |
|---|---|---|---|---|---|---|---|---|
| | of | (n = 31) | | | (# of | (n = 9) | | |
| Method | analyte | R | M | T | permutation) | R | M | T |
| PLS-DA | 10 | 62 | 73 | 68 | 0.8 (500) | 60 | 75 | 67 |
| RF | 17 | | | | 11 (100) | | | |
| PAM | 64 | 75 | 80 | 77 | 1 (100) | 60 | 60 | 56 |
| SVM | 5 | | | | 13 (100) | | | |
| Ttest | 13 | 69 | 80 | 74 | 7.5 (1000) | 80 | 100 | 89 |

Three of the five methods yielded a marginal to significant classifier using analyte lists comprising between 10 and 64 markers. CV accuracies ranged from 68 to 74% and holdout accuracies between 56 and 89%. Thirteen analytes were selected by all 3 methods (Table 5). Random Forest (RF) and Support Vector Machine (SVM) did not yield a significant classifier.

Similarly, models were compared using only the 3 conventional glycemic markers (glucose, fructosamine, HbA1c). Four of the 5 methods yielded significant classifiers. For Partial Least Square Discriminate Analysis (PLSDA) and PAM, the holdout and CV accuracies (44%-56% for holdout and 68% for CV accuracies) were worse with the conventional markers. But RF and SVM did yield a significant classifier using conventional markers. The Ttest did not yield a significant classifier using conventional markers. Thus, for the rosiglitazone or metformin-treated subjects, whether the metabolomic analytes offered an advantage over the conventional markers depended on the classification method in use.

(b) Individual Drug Fingerprint

The goal of this study was to find a set of analytes that can predict patient response to a specific oral therapy: rosiglitazone, metformin or glyburide. Since data was only available for ˜21 subjects per oral therapy, all subjects were included in the cross-validation group. Significant classifiers were obtained for predicting rosiglitazone outcomes using metabolomic data prior to treatment (Table 6).

TABLE 6
Classification results for Rosiglitazone treated subjects.

| | Number | CV Accuracy | | | % Permutation |
|---|---|---|---|---|---|
| | of | (n = 21) | | | (# of |
| Method | analytes | NR | RS | T | permutation) |
| PLS-DA | 55 | 83 | 44 | 67 | 8 (500) |
| RF | 66 | 92 | 67 | 81 | 4 (100) |
| PAM | 67 | 92 | 56 | 76 | 5 (100) |
| SVM | 20 | 100 | 56 | 81 | 4 (100) |
| Ttest | 3 | 75 | 89 | 81 | 7.5 (1000) |

RS = Responder, NR = non-responder, T = overall accuracy.

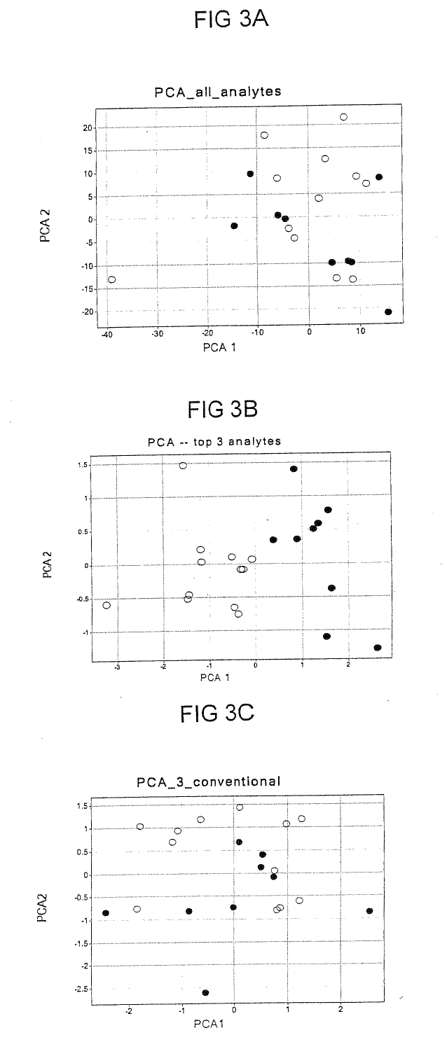

CV accuracies ranged from 67% to 81% using 3 to 67 analytes. We noted that a classifier built from 3 analytes using T-test/Majority Vote had a cross validation accuracy of 81%. The 3 analytes were also included in the list of features picked by the other four classifiers. These 3 analytes (urine citrate, serum methyl histidine, and serum IL-8) showed good separation evident between the responder and non-responder groups (FIG. 3B), whereas using 1,306 analytes included in this analysis does not indicate separation of the two groups (FIG. 3A). In comparison, the CV accuracies were worse using the three conventional glycemic biomarkers (glucose, fructosomine, HbA1c) than using the set of metabolomic analytes. This was consistent with the PCA plot of the three conventional biomarkers alone, where there was no clear separation of responders vs non-responders (FIG. 3C).

For metformin-treated subjects, only PLS-DA and SVM yielded classifiers with a permutation percentage of less than 10% (or p<0.1). The CV accuracies were 68 and 79% respectively with 110 and 5 analytes picked by each classifier (Table 4). For glyburide-treated subjects, none of the methods yielded a significant model predicting its treatment outcome. This result is consistent with the observation in the cross-drug analysis shown above that the accuracy in classifying glyburide-treated subjects was lowest among the 3 drugs.

(c) Biological Contextualization

Even with measures of accuracy and statistical significance, it is difficult to objectively assess the performance of multiple methods without applying them in practical studies. To better understand the biological relevance, we examined whether any of the selected analytes have previously been implicated in the pathophysiology of T2DM.

Rosiglitazone, metformin and glyburide affect different biological processes through various mechanisms of action and target tissues [Ahmann, A. J. & Riddle, M. C. Postgrad. Med. 111, 32-40, 43 (2002)]. Therefore, it seems intuitive that the analytes in predictive classifier rules, if collectively predictive of a particular drug's treatment outcome, should be closely related to that drug's presumed mechanism(s) of action. This expectation is largely supported by our results.

TABLE 7
Summary of analytes with known annotation for baseline prediction of
rosiglitazone responder. The analytes were selected by at least one of the classifiers.
The redundant analytes were not included in the table.

| Platform/Analyte ID | Annotation | # of method |
|---|---|---|
| Energy metabolism | | |
| GSKNMRh6:[2.69 . . . 2.72] | Citrate | 5 |
| GSKNMRh6:[4.09 . . . 4.12] | Lactate | 2 |
| Adipogenesis and release of | | |
| adipokines | | |
| NonOmics:Leptin_levels | Leptin | 2 |
| BGNMR:3.6402 | Glycerol | 3 |
| immune or inflammatory response | | |
| HumBio:IL-12p40 | IL-12p40 | 2 |
| HumBio:IL-8 | IL-8 | 5 |
| Fatty acid induced insulin resistance | | |
| NonOmics:Insulin | Insulin | 2 |
| HumBio:PAPP_A | PAPP_A | 2 |
| LipPerc:Triglyceride_Total | Total Triglyceride | 1 |
| LipPerc:Free Fatty Acid_Total | Total Free Fatty Acid | 1 |
| Amino acid | | |
| BGNMR:7.4352 | L-Phenylalanine | 1 |
| BGNMR:2.3792 | L-Proline | 1 |
| BGNMR:6.8849 | L-Tyrosine | 1 |
| BGNMR:0.9902 | L-Valine | 3 |
| GSKNMRh6:[1.76 . . . 1.77] | Lysine | 3 |
| GSKNMRh0:[1.02 . . . 1.03] | isoleucine | 2 |
| GSKNMRh6:[6.89 . . . 6.92] | Tyrosine | 1 |
| BGNMR:3.1408 | 1-Methyl-L-Histidine | 5 |
| GSKNMRh6:[0.95 . . . 0.98] | Leucine | 2 |
| Other | | |
| GSKNMRh0:[8.67 . . . 8.68] | Nicotinamide | 2 |
| GSKNMRh6:[8.92 . . . 8.93] | Nicotinate | 1 |
| GSKNMRh0:[6.67 . . . 6.70] | N-methyl-2-pyridone-5- | 1 |
| | carboxamide | |
| NonOmics:Uric_acid | Uric acid | 2 |
| NonOmics:Weight(Weight_Units_~_WTU_K) | Weight | 2 |
| HumBio:DPP-IV_Activity | DPP-IV_Activity | 2 |
| HumBio:NT-pBNP | NT-pBNP | 2 |
| BGPolar:LC-MS-Polar- | Hydroxyxanthine M | 2 |
| Metabolites:169_0271 | (M + H) | |
| GSKNMRh6:[1.09 . . . 1.12] | 2-keto-3-methyl-N- | 4 |
| | valerate | |
| GSKNMRh6:[1.26 . . . 1.28] | 3-hydroxyisovalerate | 3 |
| LipPerc:Sphingomyelin_Total | Sphingomyelin | 1 |
| LipPerc:Phosphatidylethanolamine_Total | Phosphatidylethanolamine | 1 |
| LipPerc:Lysophosphatidylcholine_Total | Lysophosphatidylcholine | 1 |
| LipPerc:Cholesterol Ester_Total | Cholesterol Ester | 1 |
| LipPerc:Diglyceride_Total | Diglyceride | 1 |
| LipPerc:Cholesterol Ester_16:0 | LipPerc:Cholesterol | 1 |
| | Ester_16:0 | |
| LipPerc:Phosphatidylcholine_20:2n6 | LipPerc:Phosphatidylcholine_20:2n6 | 3 |
| LipPerc:Phosphatidylcholine_20:3n6 | LipPerc:Phosphatidylcholine_20:3n6 | 1 |
| LipPerc:Phosphatidylcholine_20:4n3 | LipPerc:Phosphatidylcholine_20:4n3 | 2 |
| LipPerc:Phosphatidylcholine_22:5n3 | LipPerc:Phosphatidylcholine_22:5n3 | 4 |
| LipPerc:Phosphatidylcholine_Total | LipPerc:Phosphatidylcholine_Total | 1 |
| LipPerc:Phosphatidylethanolamine_% n7 | LipPerc:Phosphatidylethanolamine_% n7 | 1 |
| LipPerc:Sphingomyelin_% n9 | LipPerc:Sphingomyelin_% n9 | 1 |
| LipPerc:Triglyceride_20:4n3 | LipPerc:Triglyceride_20:4n3 | 2 |
| LipQuant:Cholesterol Ester_20:5n3 | LipQuant:Cholesterol | 1 |
| | Ester_20:5n3 | |
| LipQuant:Free Fatty Acid_20:1n9 | LipQuant:Free Fatty | 2 |
| | Acid_20:1n9 | |
| LipQuant:Lysophosphatidylcholine_20:3n6 | LipQuant:Lysophosphatidylcholine_20:3n6 | 2 |
| LipQuant:Phosphatidylcholine_20:3n6 | LipQuant:Phosphatidylcholine_20:3n6 | 1 |
| LipQuant:Phosphatidylcholine_20:4n3 | LipQuant:Phosphatidylcholine_20:4n3 | 1 |
| LipQuant:Phosphatidylcholine_dm18:0 | LipQuant:Phosphatidylcholine_dm18:0 | 1 |
| LipQuant:Phosphatidylcholine_n9 | LipQuant:Phosphatidylcholine_n9 | 1 |
| LipQuant:SecondConMed | LipQuant:SecondConMed | 1 |
| GSKNMRh0:[0.89 . . . 0.92] | s panthothenate 0.9 | 1 |
| | (up), t 0.89 up | |
| | ethylmalonate | |
| BGPolar:LC-MS-Polar- | Sphingosyl- | 1 |
| Metabolites:466_2491 | phosphocholine M + 1 | |
| | (M + H) | |
| BGPolar:LC-MS-Polar- | threo-3-Hydroxy-L- | 1 |
| Metabolites:260_2672 | aspartate M (M + H) | |

For rosiglitazone responder prediction, among the 74 analytes identified by at least one method and with known annotation (Table 7), the majority is involved in the biological processes affected by rosiglitazone: increased lipogenesis in adipose tissue and increased insulin sensitivity and signaling in muscle and liver [Stumvoll, M. & Haring, H. U. Glitazones: clinical effects and molecular mechanisms. Ann. Med. 34, 217-224 (2002)]. Examples include: energy metabolism (e.g., citrate, lactate), adipogenesis and release of adipokines (e.g., glycerol, leptin), immune or inflammatory response (IL-8, IL-12p40), fatty acid-induced insulin resistance in liver or muscle (total free fatty acid, insulin, PAPP-A, total TG, and glycerol), and amino acid metabolism (Ile, Leu, Val, Pro, His, Tyr, Phe, Lys etc.). Also, quite a few analytes (such as cholesterol ester, diglyceride, nicotinamide, etc) were not implicated in T2DM or mechanism of PPAR-γ agonists.

TABLE 8
Summary of analytes with known annotation for baseline prediction of
metformin responder. The analytes were selected by at least one of the classifiers.
The redundant analytes were not included in the table.

| Platform/Analyte ID | Annotation | # of Method |
|---|---|---|
| lipid related | | |
| NonOmics:Free_fatty_acid | Free fatty acid | 1 |
| NonOmics:Cholesterol | Cholesterol | 1 |
| NonOmics:Apolipoprotein_B | Apolipoprotein_B | 1 |
| LipPerc:Diglyceride_% MUFA | Diglyceride_% MUFA | 1 |
| LipPerc:Triglyceride_% PUFA | Triglyceride_% PUFA | 1 |
| LipPerc:Phosphatidylcholine_% PUFA | Phosphatidylcholine_% PUFA | 1 |
| Amino acids | | |
| GSKNMRh0:[0.95 . . . 0.98] | 2 d 0.96, 0.97 leucine | 1 |
| BGPolar:LC-MS-Polar- | 5-Oxoproline Related Adduct | 1 |
| Metabolites:227_1998 | (M) | |
| GSKNMRh0:[0.98 . . . 1.01] | d 0.99 valine (low level t 2 | 1 |
| | aminobutyrate) | |
| BGPolar:LC-MS-Polar- | d5-Phenylalanine M + 1 (M + H) | 1 |
| Metabolites:228_1822 | | |
| GSKNMRh0:[2.42 . . . 2.44] | Glutamine | 1 |
| BGPolar:LC-MS-Polar- | L-Alanine Related Fragment | 1 |
| Metabolites:90_0969 | (M) | |
| BGNMR:2.1168 | L-Glutamine M (b-CH2) | 1 |
| BGPolar:LC-MS-Polar- | L-Tyrosine M (M + H) | 1 |
| Metabolites:182_0316 | | |
| GSKNMRh0:[3.06 . . . 3.08] | methyl histidine | 1 |
| BGPolar:LC-MS-Polar- | Thr, Glu M (M + H) | 1 |
| Metabolites:361_2213 | | |
| Others | | |
| BGNMR:2.3152 | 3-Hydroxybutyric acid Other (b- | 1 |
| | CH2) | |
| NonOmics:Diastolic_blood_pressure | Diastolic_blood_pressure | 1 |
| GSKNMRh0:[2.50 . . . 2.59] | Citrate | 1 |
| BGPolar:LC-MS-Polar- | D-Glucose M (M + H) | 1 |
| Metabolites:317_0321 | | |
| BGPolar:LC-MS-Polar- | DTT M (M + H) | 1 |
| Metabolites:255_0358 | | |
| GSKNMRh0:[3.96 . . . 3.98] | Hippurate | 1 |
| HumBio:L-Selectin | L-Selectin | 1 |
| NonOmics:Inorganic_phosphate | Inorganic_phosphate | 1 |
| LipPerc:Cholesterol Ester_14:0 | LipPerc:Cholesterol Ester_14:0 | 1 |
| LipPerc:Cholesterol Ester_14:1n5 | LipPerc:Cholesterol | 1 |
| | Ester_14:1n5 | |
| LipPerc:Diglyceride_% n6 | LipPerc:Diglyceride_% n6 | 1 |
| LipPerc:Diglyceride_% n9 | LipPerc:Diglyceride_% n9 | 1 |
| LipPerc:Diglyceride_16:1n7 | LipPerc:Diglyceride_16:1n7 | 1 |
| LipPerc:Diglyceride_18:1n9 | LipPerc:Diglyceride_18:1n9 | 1 |
| LipPerc:Diglyceride_18:2n6 | LipPerc:Diglyceride_18:2n6 | 1 |
| LipPerc:Free Fatty Acid_22:1n9 | LipPerc:Free Fatty | 1 |
| | Acid_22:1n9 | |
| LipPerc:Lysophosphatidylcholine_% n7 | LipPerc:Lysophosphatidylcholine_% | 1 |
| | n7 | |
| LipPerc:Phosphatidylcholine_% n6 | LipPerc:Phosphatidylcholine_% | 1 |
| | n6 | |
| LipPerc:Phosphatidylcholine_% n9 | LipPerc:Phosphatidylcholine_% | 1 |
| | n9 | |
| LipPerc:Phosphatidylcholine_18:1n9 | LipPerc:Phosphatidylcholine_18:1n9 | 1 |
| LipPerc:Phosphatidylethanolamine_% | LipPerc:Phosphatidylethanolamine_% | 1 |
| n6 | n6 | |
| LipPerc:Phosphatidylethanolamine_dm18:1n7 | LipPerc:Phosphatidylethanolamine_dm18:1n7 | 1 |
| LipPerc:Sphingomyelin_14:1n5 | LipPerc:Sphingomyelin_14:1n5 | 1 |
| LipPerc:Sphingomyelin_18:0 | LipPerc:Sphingomyelin_18:0 | 1 |
| LipPerc:Triglyceride_% n6 | LipPerc:Triglyceride_% n6 | 1 |
| LipPerc:Triglyceride_18:2n6 | LipPerc:Triglyceride_18:2n6 | 1 |
| LipQuant:Cholesterol Ester_14:0 | LipQuant:Cholesterol | 1 |
| | Ester_14:0 | |
| LipQuant:Cholesterol Ester_14:1n5 | LipQuant:Cholesterol | 1 |
| | Ester_14:1n5 | |
| LipQuant:Cholesterol Ester_18:4n3 | LipQuant:Cholesterol | 1 |
| | Ester_18:4n3 | |
| LipQuant:Diglyceride_16:1n7 | LipQuant:Diglyceride_16:1n7 | 1 |
| LipQuant:Free Fatty Acid_22:1n9 | LipQuant:Free Fatty | 1 |
| | Acid_22:1n9 | |
| LipQuant:Lysophosphatidylcholine_n7 | LipQuant:Lysophosphatidylcholine_n7 | 1 |
| LipQuant:Phosphatidylethanolamine_18:3n3 | LipQuant:Phosphatidylethanolamine_18:3n3 | 2 |
| LipQuant:Triglyceride_18:2n6 | LipQuant:Triglyceride_18:2n6 | 1 |
| LipQuant:Triglyceride_18:3n3 | LipQuant:Triglyceride_18:3n3 | 1 |
| LipQuant:Triglyceride_dm16:0 | LipQuant:Triglyceride_dm16:0 | 1 |
| LipQuant:Triglyceride_n3 | LipQuant:Triglyceride_n3 | 1 |
| LipQuant:Triglyceride_n6 | LipQuant:Triglyceride_n6 | 1 |
| LipQuant:Triglyceride_PUFA | LipQuant:Triglyceride_PUFA | 1 |
| BGNMR:1.7008 | L-Leucine M (CH2) | 2 |
| NonOmics:MCH_concentration | MCH | 1 |
| GSKNMRh0:[8.89 . . . 8.92] | N1-methylnicotinamide | 1 |
| NonOmics:Sodium | Sodium | 1 |
| BGPolar:LC-MS-Polar- | Sphingosine M + 1 (M + H) | 1 |
| Metabolites:301_2536 | | |
| BGPolar:LC-MS-Polar- | Sphingosyl-phosphocholine M | 1 |
| Metabolites:465_2488 | (M + H) | |

For metformin responder prediction, the 72 markers identified by at least one method (and with known annotation) were similarly enriched in those biological processes potentially involved in metformin action (Table 8). Metformin is thought to produce an energy ‘sink’ in the liver possibly mediated via the energy sensing AMP kinase system, resulting in both decreased hepatic lipogenesis and gluconeogenesis [Kirpichnikov, D., McFarlane, S. I., & Sowers, J. R. Metformin: an update. Ann. Intern. Med. 137, 25-33 (2002)]. Thus many of the highlighted analytes were lipids and most of the non-omic markers were also lipid-related, such as apoB, cholesterol and free fatty acid. Additionally, another large component of the metformin responder marker list included amino acids, which are essential substrates for gluconeogenesis.

For cross-drug fingerprints, analytes by definition will be less revealing of specific drug class-related mechanisms, because the classification engines must select what is common to the two or more of the drugs. These cross-drug analytes are more likely to reflect markers of glucose-lowering per se and less likely to identify markers indicative of either a physiological subtype (e.g. insulin resistance) or related to a treatment-specific mechanism of action (e.g. increased adipose lipogenesis).

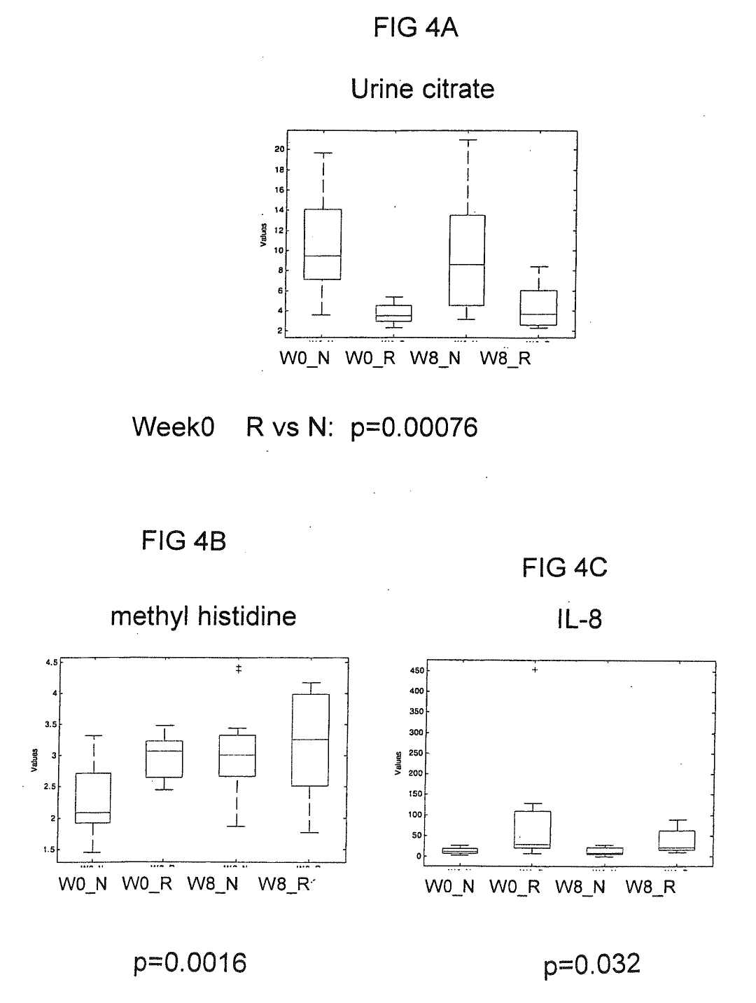

The three analytes measured at week 0 that were most predictive of week 8 rosiglitazone treatment were serum IL-8, serum methyl histidine measured by NMR (with medium confidence in annotation) and citrate in urine (with high confidence in annotation). Each of the three analytes grouped by their treatment response at week 0 and week 8 is shown in the boxcharts at FIGS. 4A-4C.

The level of urine citrate at baseline was significantly lower in responders than non-responders (p<0.001). The 8 week treatment did not change the level of urine citrate in non-responder subjects. However, it did increase urine citrate (not statistically significant) in the responder group (FIG. 4A). Citrate may play a critical role in cataplerosis (the export of mitochondrial intermediates into the cytosol and in the induction of fatty acid-derived signaling molecules) and glucose-regulated insulin release [Flamez, D. et al., Diabetes 51, 2018-2024 (2002)]. Because citrate was not quantified in plasma or liver, it is hard to pinpoint the actual biochemical context for the change in this metabolite. It could be related to uncontrolled gluconeogenesis in liver tissue. However, it cannot be ruled out that the higher citrate excretion might also depend on increased citrate production in renal tubular cells or from reduced citrate re-absorption from the tubular fluid due to glucose overflow. Increased excretion of urinary citrate has been observed in previous NMR studies of diabetic human subjects [Zuppi, C. et al. Influence of feeding on metabolite excretion evidenced by urine 1H NMR spectral profiles: a comparison between subjects living in Rome and subjects living at arctic latitudes (Svaldbard). Clin. Chim. Acta 278, 75-79 (1998); Salek, R. M. et al. A Metabolomic Comparison Of Urinary Changes In Type 2 Diabetes In Mouse, Rat And Man., Physiol Genomics (2006)]. Serum methyl histidine at baseline was higher in responders than non-responders (p=0.0016) (FIG. 4B). In a diabetic state, many alternative sources of energy are used when tissue glucose concentration and utilization are low. These include enhanced degradation of proteins and amino acids [Dice, J. F. & Walker, C. D. Ciba Found. Symp. 331-350 (1979)]. Altered excretion of methyl histidines are well established indicators of the degree of degradation of skeletal muscle proteins [Chinkes, D. L., Curr. Opin. Clin. Nutr. Metab Care 8, 534-537 (2005); Young, V. R. & Munro, H. N. Fed. Proc. 37, 2291-2300 (1978)]. The results in the present study suggested that subjects with a higher degree of protein degradation in skeletal muscle were more likely to respond to rosiglitazone treatment.

Serum IL-8 at baseline was higher in responders than non-responders (p=0.032) (FIG. 4C). IL-8 is an important cytokine in the inflammatory process. It is stimulated by high glucose concentrations in endothelial cells in vitro and has chemotactic activity for polymorphonuclear neutrophils (playing an important role in the pathogenesis of chronic complications of diabetes), as well as for T-lymphocyte and smooth muscle cells. Serum IL-8 level was reported to markedly increase in diabetic patients [Zozulinska, D., et al., Diabetologia 42, 117-118 (1999)]. It was observed it in this study and has also been reported in the literature that one of the effects of rosiglitazone treatment is to reduce apparent inflammation associated with obesity and diabetes [Belvisi, M. G., Hele, D. J., & Birrell, M. A., Eur. J. Pharmacol. 533, 101-109 (2006)]. Thus, it seems consistent that subjects with higher IL-8 levels were more responsive to rosiglitazone treatment.

### Example 3

### Early Indicators of Drug Treatment Response: the Cross-Drug or Individual Drug Fingerprint at Week 4 which is Predictive of Week 8 Treatment Response

The goal of this study was to identify “early indicator” analytes measured at week 4 of treatment (after being adjusted for week 0 baseline values) that could be used to predict drug response at week 8 of treatment. Similar to the analysis of baseline analytes predictive of treatment response, the exercise was repeated for analytes measured at 4 weeks. Conventional glycemic markers (glucose, fructosamine and HbA1c) were again excluded from the analysis.

(a) Cross-Drug Fingerprint

Classification analysis was applied to 4 week data from 75 clinical subjects who were treated with one of the 3 study drugs or placebo, seeking response markers common to all three drugs. PAM and SVM did not yield significant classifiers (Table 9). As shown in Table 9, the number of analytes indicates the optimal number that maximized prediction accuracy in cross-validation. The percentage of permutation is the percent of permutation runs that had better CV accuracy than the original CV accuracy. The number in brackets indicates the number of permutation runs which was method dependant.

TABLE 9
Cross-drug classification results.

| | Number | CV Accuracy | | | | | % Permutation | Holdout Accuracy | | | | |
|---|---|---|---|---|---|---|---|---|---|---|---|---|
| | of | (n = 58) | | | | | (# of | (n = 17) | | | | |
| Method | analyte | P | R | M | G | T | permutation) | P | R | M | G | T |
| PLS-DA | 50 | 83 | 56 | 47 | 60 | 60 | 2.8 (500) | 33 | 40 | 75 | 90 | 59 |
| RF | 28 | 92 | 56 | 53 | 73 | 67 | 8 (100) | 67 | 60 | 75 | 40 | 59 |
| PAM | | | | | | | 68 (100) | | | | | |
| SVM | | | | | | | 66 (250) | | | | | |
| Ttest | 79 | 75 | 62 | 73 | 73 | 71 | 7.2 (1000) | 67 | 80 | 75 | 60 | 71 |

P = placebo, R = rosiglitazone, M = metformin, G = glyburide, T = overall accuracy.

The number of analytes ranged from 28 for RF to 79 for the Ttest method. The overall CV accuracies ranged from 60 to 71% and the holdout accuracies from 59 to 71%. The 3 methods that yielded marginal or significant results selected a total of 98 different analytes as being important in the classification. A PCA plot using 50 analytes selected by at least two methods (Table 10) did offer discriminating power between the two groups of subjects.

TABLE 10
Predictive fingerprint at week 4. The number in the method column
indicates how many methods selected the analyte.

| | | |
|---|---|---|
| Platform: Analyte ID | Annotation | Method |

Week 4 cross-drug fingerprint, 50 analytes picked by at least two methods

| | | |
|---|---|---|
| Lipid:615_1394 | 34:2 DG M + Na (M) | 3 |
| Lipid:815_1527 | 24:1 SM M + H (M + 2) | 3 |
| NMR:1.9248 | L-Lysine and/or Acetate Other (b-CH2/ | 3 |
| | CH3) | |
| Polar:LC-MS-Polar-Metabolites:185_1167 | N/A | 3 |
| Polar:LC-MS-Polar-Metabolites:371_2004 | 5-Oxoproline Related Dimer (M) | 3 |
| NMRh0:[6.65 . . . 6.67] | low level unassigned peak | 3 |
| HumBio:E-selectin | E-selectin | 3 |
| LipPerc:Free Fatty Acid_22:4n6 | Free Fatty Acid_22:4n6 | 3 |
| LipPerc:Phosphatidylethanolamine_% dm | Phosphatidylethanolamine_% dm | 3 |
| Lipid:575_2021 | TG MS fragment M + H (M) | 2 |
| Lipid:576_2021 | TG MS fragment M + H (M + 1) | 2 |
| Lipid:643_1477 | 18:1/18:1 DG M + Na (M) | 2 |
| Lipid:799_1503 | 23:1 SM M + H (M) | 2 |
| Lipid:820_1953 | 18:1/16:1/14:0 TG M + NH4 (M) | 2 |
| Lipid:821_1953 | 18:1/16:1/14:0 TG M + NH4 (M + 1) | 2 |
| Lipid:822_1997 | 18:1/16:0/14:0 TG M + NH4 (M) | 2 |
| Lipid:825_1949 | 18:2/16:0/14:0 TG M + Na (M) | 2 |
| Lipid:826_1949 | 18:2/16:0/14:0 TG M + Na (M + 1) | 2 |
| Lipid:835_1525 | 24:1 SM M + Na (M) | 2 |
| Lipid:846_1969 | 18:2/18:1/14:0 TG M + NH4 (M) | 2 |
| Lipid:847_1969 | 18:2/18:1/14:0 TG M + NH4 (M + 1) | 2 |
| Lipid:848_2011 | 18:2/16:0/16:0 TG M + NH4 (M) | 2 |
| Lipid:849_2011 | 18:2/16:0/16:0 TG M + NH4 (M + 1) | 2 |
| Lipid:924_2037 | 20:4/18:1/18:1 TG M + NH4 (M) | 2 |
| NMR:2.1424 | L-Glutamine M (b-CH2) | 2 |
| Polar:LC-MS-Polar-Metabolites:122_0173 | N/A | 2 |
| Polar:LC-MS-Polar-Metabolites:170_2783 | N/A | 2 |
| Polar:LC-MS-Polar-Metabolites:211_2797 | N/A | 2 |
| Polar:LC-MS-Polar-Metabolites:221_0310 | N/A | 2 |
| Polar:LC-MS-Polar-Metabolites:363_2192 | Glu, Cys M + H (M) | 2 |
| Polar:LC-MS-Polar-Metabolites:585_2488 | N/A | 2 |
| Polar:LC-MS-Polar-Metabolites:784_1235 | N-Acetylneuraminate Related Dimer (M) | 2 |
| NMRh0:[2.50 . . . 2.59] | citrate | 2 |
| NMRh0:[2.66 . . . 2.69] | citrate | 2 |
| NMRh0:[6.67 . . . 6.70] | N-methyl-2-pyridone-5-carboxamide | 2 |
| NMRh0:[8.31 . . . 8.32] | low level unassigned peak | 2 |
| HumBio:Glycerol | Glycerol | 2 |
| HumBio:I-CAM | I-CAM | 2 |
| HumBio:L-Selectin | L-Selectin | 2 |
| HumBio:MMP-9_2 | MMP-9_2 | 2 |
| LipPerc:Cholesterol Ester_20:3n6 | Cholesterol Ester_20:3n6 | 2 |
| LipPerc:Diglyceride_20:0 | Diglyceride_20:0 | 2 |
| LipPerc:Free Fatty Acid_22:0 | Free Fatty Acid_22:0 | 2 |
| LipPerc:Lysophosphatidylcholine_22:5n3 | Lysophosphatidylcholine_22:5n3 | 2 |
| LipPerc:Phosphatidylethanolamine_dm16:0 | Phosphatidylethanolamine_dm16:0 | 2 |
| LipPerc:Phosphatidylethanolamine_dm18:0 | Phosphatidylethanolamine_dm18:0 | 2 |
| LipQuant:Phosphatidylethanolamine_22:4n6 | Phosphatidylethanolamine_22:4n6 | 2 |
| LipQuant:Sphingomyeln_nmol lipid class | Sphingomyelin_nmol lipid class per g | 2 |
| per g sample | sample | |
| NonOmics:ALPC3 | ALPC3 | 2 |
| NonOmics:VLDL | VLDL | 2 |

Week 4 glyburide fingerprint, 10 analytes picked by at least 2 methods

| | | |
|---|---|---|
| Lipid:799_1503 | 23:1 SM M + H (M) | 4 |
| NMR:7.3265 | L-Phenylalanine M (H-2/H-6) ** | 4 |
| NMR:7.32 | L-Phenylalanine M (H-2/H-6) | 3 |
| Polar:LC-MS-Polar-Metabolites:185_1167 | N/A | 3 |
| Polar:LC-MS-Polar-Metabolites:189_1106 | N/A | 3 |
| NMR:2.6545 | Citrate Other (CH2) | 2 |
| NMR:4.1072 | Lactate Q (CH) | 2 |
| NMR:4.12 | Lactate and/or L-Proline Other (CH/a- | 2 |
| | CH) | |
| Polar:LC-MS-Polar-Metabolites:169_1108 | N/A | 2 |
| Polar:LC-MS-Polar-Metabolites:377_0351 | N/A | 2 |

In contrast to results using metabolomic data, all 5 methods yielded significant models using the 3 conventional glycemic markers (data not shown). For the methods that yielded significant classifiers using metabolomic data, the accuracies using the conventional markers were generally better. This is not unexpected given that glucose and fructosamine are relatively early response markers and might be expected at 4 weeks to correlate highly with their corresponding values at 8 weeks.

(b) Individual Drug Fingerprint

The 5 classification methods were applied to the problem of predicting response at week 8 for the subjects treated with a single drug, using metabolomic data at week 4 that was adjusted for baseline week 0 values.

For glyburide treatment, all methods except RF gave significant classifiers with minimal marker lists ranging in size from 2 to 19 analytes and CV accuracies ranging from 65 to 90% (Table 11).

TABLE 11
Classification results for glyburide treated subjects.

| | Number | CV Accuracy | | | % Permutation |
|---|---|---|---|---|---|
| | of | (n = 21) | | | (# of |
| Method | analytes | NR | RS | T | permutation) |
| PLS-DA | 10 | 88 | 50 | 65 | 5.6 (500) |
| RF | | | | | 24 (100) |
| PAM | 19 | 62 | 92 | 80 | 2 (100) |
| SVM | 5 | 75 | 92 | 85 | 3 (100) |
| T-test | 2 | 88 | 92 | 90 | 2.3 (1000) |

RS = Responder, NR = non-responder, T = overall accuracy.

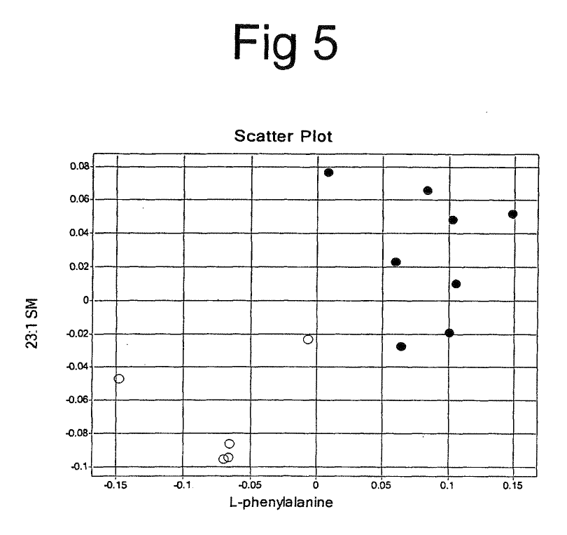

The 10 analytes picked by at least 3 methods are listed in Table 10. Good separation between the responder and non-responder groups is evident from the plot of 2 analytes, L-phenylalanine and sphingomyelin, with the responders segregating towards the upper right of the plot (FIG. 5). Results for the same classification using conventional markers were better than the corresponding results from metabolomic data for most methods with the exception of the Ttest classifier. The two analytes picked by Ttest were serum 23:1 sphingomyelin (SM) and L-phenylalanine. SM is a type of lipid involved in facilitating neural transmission in animals. The implication of sphingomyeline and L-phenylalanine in the glyburide response is unclear.

For rosiglitazone, only RF and PLS-DA gave significant classifiers. Using the three conventional markers, the results were better or equivalent to the corresponding results using all metabolomic data. For metformin, none of the classifiers yielded significant models.

The multivariate methods used to identify the classifier rules have unique value in identifying analytes that do not necessarily declare themselves in more conventional statistical analyses, such as correlation or univariate change approaches. Many on the classifier lists are not significantly correlated with the common clinical endpoints nor changed by treatment with a statistically significant mean fold change. However, when used in a relational way with the other markers within the list, they may unmask other non-obvious elements of disease biology or treatment effect.

Each analytical method generated a different set of predictive fingerprints. Other studies have also shown that the discriminatory features can vary significantly from one data mining technique to another [Li, L. et al. Artif. Intell. Med. 32, 71-83 (2004)]. It is interesting to probe whether the results from multiple methods provide any advantage over a single method. Another step that will be required is to combine/synthesize the results from multiple methods in a way that extracts the value inherent in multiple analyses of the same datasets. Currently, a composite fingerprint list could be generated from the multiple lists, and the composite list then be filtered to eliminate markers that cannot be identified or measured in a clinical chemistry assay. The fingerprint list could also be filtered for biological content. Once this filtering is complete, one or more classifiers/prediction rules might be rebuilt using the filtered list of analytes. This rule could then be tested in validation studies.

This biological contextualization is focused on the baseline analyte groupings. Many of the analytes included in the baseline classifier lists for all 3 drugs, for rosiglitazone and metformin separately, or for the two drugs combined, appear intuitively to be related to lipid or energy metabolism, insulin biology (e.g. IGF-1), or fat cell biology (adipokines such as leptin and lipids). Further analyses are required in order to develop fuller contextualization regarding classifier analytes lists.

It is somewhat surprising that attempts failed to identify classifier rules for glyburide using baseline analytes. This suggests that prediction of glyburide response may not depend on disease severity or other readily discernible metabolite or lipid patterns. It also suggests that the analytes detected on the “open profiling” metabolomic platforms do not include strong baseline correlates for insulin reserve—a presumed requirement for effective glyburide action. Understanding this will require further exploration. In contrast, the identification of analyte lists for rosiglitazone alone, as well as for combined data of rosiglitazone and metformin, suggests that baseline analytes may well be useful in defining insulin resistance and/or identifying the relative potential within that individual for creating energy or metabolic ‘sink’, presumed therapeutic sequelae of these two agents.

The analysis of 4-week markers for therapeutic efficacy provided herein suggests that FPG, fructosamine and HbA1c might be used with greater precision and with relational rule building to more precisely identify efficacy at 4 weeks. However, these findings also suggest that there are likely other markers at 4 weeks that also define drug efficacy and that these might have unique utility. In addition, understanding the differences in the 4-week predictive markers utilized to predict, for example, glyburide vs rosiglitazone efficacy may point to a marker signature that can differentiate glucose-lowering due to increased insulin mass action (obtained with a secretagogue such as glyburide or incretin such as GLP-1) versus an insulin-sensitizer such as a PPAR-γ agonist. Such pharmacometabolomic differentiation, in turn, may one day be applied in the clinical setting to ascertain whether specific drug mechanisms are operative in achieving efficacy within an individual patient.

Individually, the markers discussed in this study have potential biologic plausibility in the pathogenesis of T2DM. When taken as a whole, via multivariate models, these markers are reflect a more synthesized view of biological state changes.

All publications cited in this specification are incorporated herein by reference. While the invention has been described with reference to a particularly preferred embodiment, it will be appreciated that modifications can be made without departing from the spirit of the invention. Such modifications are intended to fall within the scope of the appended claims.

1. A method for predicting treatment response of a type II diabetes patient to rosiglitazone, the method comprising:
obtaining at least one sample from a patient having type II diabetes;
analyzing biomarkers predictive of a patient who will respond to treatment with rosiglitazone, said biomarkers identified in at least one classification analyses selected from the group consisting of a majority-vote classifier and a support vector machine (SVM) classifier.

2. The method according to claim 1, wherein the majority-vote classifier is a t-test.

3. The method according to claim 1, wherein the biomarkers are identified in both a majority-vote and a support vector machine (SVM) analysis.

4. The method according to claim 1, wherein the biomarkers comprise interleukin-8, histidine, citrate.

5. The method of claim 4, wherein the biomarkers further comprise those selected from the group consisting of lactate, glycerol, leptin, IL-12p40, PAI-1, total free fatty acid, insulin, IGF-1, PPAP-A, total TG, glycerol, and amino acids.

6. A method for predicting treatment response of a type II diabetes patient to rosiglitazone, the method comprising:
obtaining at least one sample from a patient having type II diabetes;
analyzing biomarkers from the patient having type II diabetes comprising at least interleukin-8, histidine and citrate, said biomarkers identified in at least a majority-vote classifier analyses and a support vector machine (SVM) classifier analysis.

7. The method according to claim 6, wherein the biomarkers are further analyzed in one or more additional classification analysis selected from the group consisting of a centroid classifier, a regression-based classifier, and a tree-based classifier.

8. The method of claim 6, wherein the biomarkers are at least 80% predictive of response at 8 weeks for a patient prior to rosiglitazone treatment.

9. The method of claim 6, wherein the biomarkers are serum IL-8 and serum histidine.

10. The method of claim 9, wherein serum IL-8 levels are significantly higher in patients who will respond to rosiglitazone as compared to non-responders.

11. The method of claim 9, wherein serum histidine levels are significantly higher in patients who will respond to rosiglitazone as compared to non-responders.

12. The method of claim 6, wherein the biomarker is citrate in urine.

13. The method of claim 12, wherein urine citrate levels are significantly lower in patients who will respond to rosiglitazone as compared to non-responders.

14. A method for predicting treatment response of a type II diabetes patient to glyburide at 8 weeks post-initiation of therapy, the method comprising:
(a) obtaining a sample from a type II diabetes patient who has been treated with glyburide for about 4 weeks; and
(b) analyzing biomarkers predictive of a patient who will respond to treatment with glyburide at 8 weeks, said biomarkers identified in at least three classification analyses selected from the group consisting of a regression-based classifier, a centroid classifier, a support vector machine (SVM), and a majority-vote-based classifier.

15. The method according to claim 14, wherein the biomarkers comprise sphingomyelin 23:1 and L-phenylalanine.

16. The method according to claim 14, wherein the biomarkers further comprise glucose, fructosamine and HbA1c.

17. The method according to claim 16, wherein the regression-based classifier is a partial least squares-discriminant analysis (PLS-DA).

18. The method according to claim 16, wherein the centroid classifier is a prediction analysis for microarrays.

19. The method according to claim 16, wherein the majority-vote-based classifier is a t-test.

20. A kit useful for predicting a type II diabetes patient response to rosiglitazone, said kit comprising:
one or more reference standards providing baseline levels of selected biomarker analytes in type II diabetes patients which are responsive to rosiglitazone,
optionally, providing one or more reference standards providing baseline levels of the selected analytes in type II diabetes patients which are non-responsive to rosiglitazone.

21. A kit useful for predicting a type II diabetes patient response to glyburide, said kit comprising:
one or more reference standards providing levels of selected biomarker analytes in type II diabetes patients which have been treated with glyburide for 4 weeks and are responsive to glyburide,
optionally, providing one or more reference standards providing baseline levels of the selected analytes in type II diabetes patients which are non-responsive to glyburide.

22. A method of predicting a subject's responsiveness to a thiazolidinedione, the method comprising:
identifying the subject as having an increased or decreased likelihood of a response to a thiazolidinedione based on analysis of one or more biomarkers comprising citrate, methyl histidine, or interleukin-8.

23. The method of claim 22, wherein the method comprises detecting citrate in a sample from the subject.

24. The method of claim 22, wherein the sample is a urine sample.

25-34. (canceled)

35. A method of predicting a subject's responsiveness to a sulfonylurea, the method comprising:
identifying the subject as having an increased or decreased likelihood of a response to a sulfonylurea based on analysis of one or more biomarkers comprising phenylalanine or 23:1 sphingomyelin.

36. The method of claim 35, wherein the subject is a subject to whom the sulfonylurea was administered prior to analysis of the one or more biomarkers.

37. The method of claim 36, wherein the sulfonylurea was administered for fewer than eight weeks.

38. The method of claim 37, wherein the sulfonylurea was administered for about four weeks.

39-49. (canceled)

50. A method of predicting a subject's responsiveness to a thiazolidinedione, the method comprising:
calculating, based on a concentration of at least one biomarker in a sample from a subject, an index having a value indicative of the likelihood of the subject responding to the thiazolidinedione, wherein the at least one biomarker comprises citrate, methyl histidine, or interleukin-8; and
displaying, transmitting, or storing the index.

51. A method of predicting a subject's responsiveness to a sulfonylurea, the method comprising:
calculating, based on a concentration of at least one biomarker in a sample from a subject, an index having a value indicative of the likelihood of the subject responding to the sulfonylurea, wherein the at least one biomarker comprises phenylalanine or 23:1 sphingomyelin; and
displaying, transmitting, or storing the index.

52-53. (canceled)

54. The method of claim 51 wherein the index is transmitted to a person in a medical industry.

55. The method of claim 51 wherein the index is transmitted to a medical insurance provider or to a physician.

56. The method of claim 50, wherein the index is transmitted prior to the medical insurance provider or the physician approving the thiazolidinedione or the sulfonylurea for the subject.

57. (canceled)

58. The method of claim 50, wherein the method comprises calculating an index based on concentrations of methyl histidine and interleukin-8 in a blood-based sample and a concentration of citrate in a sample comprising urine.

59-62. (canceled)
